(19) United States
(12) Patent Application Publication
(10) Pub. No.: US 2025/0120972 A1
(43) Pub. Date: Apr. 17, 2025

(54) PHARMACY COMPOUNDED PRESCRIPTION COMPOSITIONS FOR HAIR GROWTH

(71) Applicant: REMI MEDICAL, LLC, Henderson, NV (US)

(72) Inventor: SHAHIN SHANE MALEK, Henderson, NV (US)

(73) Assignee: REMI MEDICAL, LLC, Henderson, NV (US)

(21) Appl. No.: 18/488,375

(22) Filed: Oct. 17, 2023

(51) Int. Cl. A61K 31/506; A61K 9/08; A61K 31/203; A61K 31/58; A61K 47/10; A61K 47/26

(52) U.S. Cl. CPC A61K 31/506; A61K 9/08; A61K 31/203; A61K 31/58; A61K 47/10; A61K 47/26

(57) ABSTRACT

A physician-prescribed and compounding pharmacy-compounded minoxidil-containing hair growth composition is described, containing minoxidil, glycerin, polysorbate-80, and propylene glycol in aqueous ethanol solution at pH in a range of from 3.8 to 5.2. The composition may contain additional ingredients such as finasteride, dutasteride, retinoic acid, tretinoin, sulfuric acid, hypochlorous acid, and citric acid. The described hair growth compositions are stable against minoxidil precipitation for at least 6 months at temperature in a range of from 5° C. to 32° C., and may contain physician-prescribed minoxidil at a concentration that is in a range of from 1.2 wt % to 12 wt %, based on total weight of the composition. Various methods of preparation of such compositions are additionally described.

### FIELD

The present disclosure relates to pharmacy compounded prescription compositions for promoting hair growth.

### DESCRIPTION OF THE RELATED ART

A large number of hair growth compositions are known in the art, utilizing active ingredients for promoting hair growth of persons who suffer from hair loss.

Active ingredients that have been employed in hair growth compositions include hair nutrient vitamins, proteins, and minerals, plant-based botanicals, and minoxidil. Among these active ingredients, minoxidil has demonstrated effectiveness in promoting hair growth, and has been found highly efficacious for addressing hair loss resulting from a wide variety of conditions, such as for example androgenetic alopecia, traction alopecia, alopecia areata, tinea capitis, telogen effluvium, antigen effluvium, systemic lupus erythematosus, thyroid disorders, sex hormone imbalances, and therapeutic agent side effects from chemotherapeutic agents, beta-blockers, warfarin, levodopa, amphetamines, and other drugs. Upon topical administration, minoxidil is converted to the active form of minoxidil sulfate by the action of sulfotransferase in hair follicle tissue.

Minoxidil, despite its effectiveness, has associated formulation issues that constrain its use. Minoxidil is only slightly soluble in water, having an aqueous solubility on the order of 2.2 mg/milliliter at 25° C. and 1 atm pressure. While solubility is greater in alcohols and glycols, the use of such solvents at high concentrations has associated disadvantages of skin irritation, dryness, and itching, and other adverse dermatologic effects. Further, minoxidil solutions when stored are susceptible to undesirable precipitation of minoxidil crystals, particularly at neutral or basic pH conditions, and stability issues are therefore present.

In consequence of the foregoing considerations, the art continues to seek improvements in minoxidil-containing hair growth compositions.

### SUMMARY

The present disclosure relates to pharmacy compounded prescription compositions for promoting hair growth, and to pharmacy compounding procedures for preparing such compositions.

In one aspect, the disclosure relates to a physician-prescribed and compounding pharmacy-compounded minoxidil-containing hair growth composition, comprising minoxidil, glycerin, polysorbate-80, and propylene glycol in aqueous ethanol solution at pH in a range of from 3.8 to 5.2.

In another aspect, the disclosure relates to a hair growth composition, comprising: glycerin; propylene glycol; polysorbate-80; ethanol; acid for pH adjustment, e.g., sulfuric acid, hypochlorous acid, citric acid, or other suitable acid; minoxidil; at least one of finasteride and dutasteride; retinoic acid; and water.

In a further aspect, the disclosure relates to a hair growth composition, comprising:

| ingredient | weight percent, based on total weight of composition |
|---|---|
| water | 26.3875% |
| glycerin | 10% |
| propylene glycol | 30% |
| polysorbate-80 | 8% |
| ethanol | 20% |
| minoxidil | 5% |
| finasteride | 0.3% |
| tretinoin | 0.0125% |
| sulfuric acid | 0.3% |
| TOTAL | 100 weight percent |

A further aspect of the disclosure relates to a method of preparing a hair growth composition, comprising the sequential steps of:
(a) mixing a first amount of water with glycerin, propylene glycol, polysorbate-80, and ethanol, to form a first solution;
(b) heating the first solution under mixing conditions up to a first temperature in a range of from 70° C. to 80° C.;
(c) while maintaining the mixing conditions and heating to maintain the first temperature, adjusting pH of the first solution to pH in a range of from 4 to 5 by addition of acid, e.g., sulfuric acid, hypochlorous acid, citric acid, or other suitable acid;
(d) while maintaining the mixing conditions and heating to maintain the first temperature, adding minoxidil to the pH-adjusted first solution until the minoxidil is dissolved, to form a second solution; and
(e) terminating the heating, and while maintaining the mixing conditions, adding a second amount of water at temperature in a range of from 15° C. to 30° C. to the second solution, to yield the composition.

A still further aspect of the disclosure relates to a method of preparing a hair growth composition, comprising the sequential steps of
(a) mixing a first amount of water with glycerin, propylene glycol, polysorbate 80, and ethanol, to form a first solution, and heating the first solution under mixing conditions up to a first temperature in a range of from 70° C. to 80° C.;
(b) while maintaining the mixing conditions and heating to maintain the first temperature,
   (1) adjusting pH of the first solution, by addition of acid, e.g., sulfuric acid, hypochlorous acid, citric acid, or other suitable acid, to pH in a range of from 4 to 5 and adding minoxidil to the pH-adjusted first solution until the minoxidil is dissolved, to form a second solution; and
   (2) adding (i) at least one of finasteride or dutasteride, and (ii) retinoic acid to the second solution to form a third solution;
(c) terminating the heating of the third solution while maintaining the mixing conditions; and
(d) while maintaining the mixing conditions, adding a second amount of water at temperature in a range of from 15° C. to 30° C. to the third solution, to yield the hair growth composition.

In another aspect, the disclosure relates to a method of preparing a hair growth composition, comprising the sequential steps of
(a) mixing the following ingredients: 18 mL water, 10 mL glycerin, 30 mL propylene glycol, 8 mL polysorbate-80, and 20 mL ethanol, and continuing the mixing while heating the solution up to 75° C.;
(b) while continuing the mixing, adding sulfuric acid and minoxidil until a pH condition of 4.5 was achieved with all minoxidil dissolved in the solution;
(c) while continuing mixing, adding 300 mg finasteride and 12.5 mg of retinoic acid, until all solids were fully dissolved; and
(d) while continuing mixing, adding 8.6875 mL of room temperature (22° C.-26° C.) water, to complete the hair growth composition.

Other aspects, features and embodiments of the disclosure will be more fully apparent from the ensuing description and appended claims.

### DETAILED DESCRIPTION

The present disclosure relates to pharmacy compounded prescription compositions for promoting hair growth, and to pharmacy compounding procedures for preparing such compositions.

The hair growth compositions of the present disclosure provide stable solutions of minoxidil with no undissolved solids in which minoxidil is maintained in solution for extended periods of time, as topical compositions that are pharmacy compounded according to medical prescriptions generated based on age, gender, and duration and extent of hair loss of individual patients for whom the medical prescriptions are made. Such prescriptions generated for minoxidil-containing hair growth compositions of the present disclosure will specify a concentration of minoxidil that is in a range of from 1.2 wt % to 12 wt %, based on total weight of the composition for the specific individual patient for whom the composition is prescribed.

Based on the prescription generated by the physician, the prescription is formulated by a compounding pharmacy according to the methodology hereinafter more fully described, to provide a physician-prescribed and compounding pharmacy-compounded minoxidil-containing hair growth composition, comprising minoxidil, glycerin, polysorbate-80, and propylene glycol in aqueous ethanol solution at pH that is in a range of from 3.8 to 5.2, 4 to 5, or in other suitable pH range. Such composition is stable against minoxidil precipitation for at least 6 months at temperature in a range of from 5° C. to 32° C.

The composition may be formulated, wherein the physician-prescribed minoxidil concentration is a concentration that is in a range of from 1.2 wt % to 12 wt %, based on total weight of the composition, or that is in a range of from 2 wt % to 8 wt %, based on total weight of the composition, or that is in other suitable concentration range or at other specific concentration value. Such physician-prescribed minoxidil concentration is prescribed based on physician assessment of medical history and phenotypic and physiological characteristics of a patient, including age, gender, and extent and duration of hair loss.

The prescribing physician may be situated in any suitable medical services delivery system or location, including medical offices, hospitals, medical clinics, telemedicine facilities, online medical consultation facilities, or other systems or locations. The prescription that is generated by the prescribing physician may be given or transmitted to the patient for prescription fulfillment, or the prescription may be additionally or alternatively transmitted directly from the prescribing physician to the compounding pharmacy at which the prescription is filled.

The composition may be packaged by the compounding pharmacy for subsequent use with topical dosing instructions on or in packaging. The topical dosing instructions on or in the packaging may indicate suitable composition amount(s) and frequency of dosing, among other instructions. For example, the topical dosing instructions for the composition may indicate topical application of 1-2 mL, or more, of the composition to the locus of hair loss once daily. The packaging may include a cap or cup with a gradated volume scale, into which the composition from a container of same is poured to a specific volume level in the cap or cup for subsequent topical application, or the cap or cup may simply have a level marker indicating a proper dosage level for the topical application dose.

The composition may be formulated with any suitable ingredients described herein, wherein the weight percentages of all ingredients in formulation total to 100 weight percent. The composition ingredients may for example include any one or more of finasteride, dutasteride, retinoic acid, tretinoin, sulfuric acid, hypochlorous acid, and citric acid.

The composition may include 5α-reductase inhibitor selected from the group consisting of finasteride, dutasteride, and combinations of finasteride and dutasteride, e.g., wherein the concentration of finasteride and/or dutasteride, when present, is at concentration in a range of from 0.001 wt % to 0.5 wt %, based on total weight of the composition, or at concentration in a range of from 0.001 wt % to 0.2 wt %, based on total weight of the composition, or in other suitable range or at other specific concentration value.

Compositions of the present disclosure containing finasteride have been shown to reduce its flux into the bloodstream and to delay peak activity to 18-30 hours, significantly reducing rate of systemic absorption and thereby minimizing systemic side effects. Accordingly, the compositions of the present disclosure are effective to achieve slower absorption of actives (minoxidil, finasteride, dutasteride). Thus, for example, finasteride is controlled so that it is maintained at the dermal surface and does not enter the system of the user at a rate that would be able to cause unwanted systemic side effects. In this respect, it is to be noted that the hair follicle is not so much affected by serum dihydrotestosterone (DHT), but produces its own DHT at the follicular locus, and the maintained presence of finasteride at the follicular locus in the applied finasteride-containing composition of the present disclosure functions to reduce the formation of DHT that would otherwise occur at such locus.

Retinoic acid when present may be at concentration in a range of from 0.005 wt % to 0.9 wt %, based on total weight of the composition, or at other concentrations or in other concentration ranges.

Tretinoin when present may be at concentration in a range of from 0.001 wt % to 1 wt %, based on total weight of the composition, or may be at concentration in a range of from 0.005 wt % to 0.02 wt %, based on total weight of the composition, or may be in other concentration ranges or at other specific values of concentration.

The composition may contain acid for pH adjustment, e.g., sulfuric acid, hypochlorous acid (HOCl), citric acid, or other suitable acid as present at sufficient concentration to establish and maintain appropriate pH in the composition. The concentration of acid in the composition may for example be in a range of from 0.08 wt % to 0.6 wt %, based on total weight of the composition, or in other concentration ranges or at other specific values of concentration. In specific embodiments, the acid for pH adjustment comprises at least one of sulfuric acid and hypochlorous acid.

In various embodiments of hair growth compositions of the present disclosure, the hair growth composition may comprise: glycerin; propylene glycol; polysorbate-80; ethanol; acid for pH adjustment, e.g., sulfuric acid, hypochlorous acid (HOCl), citric acid, or other suitable acid; minoxidil; at least one of finasteride and/or dutasteride; retinoic acid; and water.

In other embodiments of the hair growth compositions of the present disclosure, the hair growth composition may comprise: glycerin; propylene glycol; polysorbate-80; ethanol; minoxidil; finasteride; tretinoin; acid for pH adjustment, e.g., sulfuric acid, hypochlorous acid (HOCl), citric acid, or other suitable acid; and water. In one specific embodiment, the composition may comprise the following composition by weight:

| | Concentration, weight percent, based on |
|---|---|
| Component | total weight of composition |
| water | 26.3875% |
| glycerin | 10% |
| propylene glycol | 30% |
| polysorbate-80 | 8% |
| ethanol | 20% |
| minoxidil | 5% |
| finasteride | 0.3% |
| tretinoin | 0.0125% |
| sulfuric acid | 0.3% |
| TOTAL | 100 weight percent. |

Compositions of the present disclosure may be prepared by compounding pharmacy compounding of the composition according to the physician prescription.

In various embodiments, the compounding pharmacy compounding of the hair growth composition may comprise the sequential steps of
(a) mixing a first amount of water with glycerin, propylene glycol, polysorbate-80, and ethanol, to form a first solution;
(b) heating the first solution under mixing conditions up to a first temperature in a range of from 70° C. to 80° C.;
(c) while maintaining the mixing conditions and heating to maintain the first temperature, adjusting pH of the first solution to pH in a range of from 4 to 5 by addition of acid for pH adjustment, e.g., sulfuric acid, hypochlorous acid (HOCl), citric acid, or other suitable acid;
(d) while maintaining the mixing conditions and heating to maintain the first temperature, adding minoxidil to the pH-adjusted first solution until the minoxidil is dissolved, to form a second solution; and
(e) terminating the heating, and while maintaining the mixing conditions, adding a second amount of water at temperature in a range of from 15° C. to 30° C. to the second solution, to yield the composition.

In various implementations of the foregoing compounding pharmacy compounding method for preparing the hair growth composition, step (d) may include addition of one or more of finasteride, dutasteride, and tretinoin, e.g., a combination of two of such ingredients such as a combination of finasteride and dutasteride, a combination of finasteride and tretinoin, or a combination of dutasteride and tretinoin, or addition of all three of such ingredients, prior to carrying out step (e).

In various other embodiments, the compounding pharmacy compounding of the composition may comprise the sequential steps of
(a) mixing a first amount of water with glycerin, propylene glycol, polysorbate 80, and ethanol, to form a first solution, and heating the first solution under mixing conditions up to a first temperature in a range of from 70° C. to 80° C.;
(b) while maintaining the mixing conditions and heating to maintain the first temperature,
   (1) adjusting pH of the first solution, by addition of acid for pH adjustment, e.g., sulfuric acid, hypochlorous acid (HOCl), citric acid, or other suitable acid, to pH in a range of from 4 to 5 and adding minoxidil to the pH-adjusted first solution until the minoxidil is dissolved, to form a second solution; and
   (2) adding (i) at least one of finasteride or dutasteride, and (ii) retinoic acid to the second solution to form a third solution;
(c) terminating the heating of the third solution while maintaining the mixing conditions; and
(d) while maintaining the mixing conditions, adding a second amount of water at temperature in a range of from 15° C. to 30° C. to the third solution, to yield the hair growth composition.

In such compounding pharmacy compounding of the hair growth composition, minoxidil is added to provide a concentration that is in a range of from 1.2 wt % to 12 wt %, based on total weight of the composition, and acid for pH adjustment, e.g., sulfuric acid, hypochlorous acid (HOCl), citric acid, or other suitable acid, is added only in a quantity required to achieve the specified pH condition of pH 4 to 5.

In yet another embodiment, wherein the hair growth composition comprises an aqueous ethanol solution of glycerin, propylene glycol, polysorbate-80, minoxidil, acid for pH adjustment, e.g., sulfuric acid, hypochlorous acid (HOCl), citric acid, or other suitable acid, finasteride, and retinoic acid, the compounding pharmacy compounding of the hair growth composition may comprise the sequential steps of:
(a) mixing the following ingredients: 18 mL water, 10 mL glycerin, 30 mL propylene glycol, 8 mL polysorbate-80, and 20 mL ethanol, and continuing the mixing while heating the solution up to 75° C.;
(b) while continuing the mixing, adding acid for pH adjustment, e.g., sulfuric acid, hypochlorous acid (HOCl), citric acid, or other suitable acid, and minoxidil until a pH condition of 4.5 is achieved with all minoxidil dissolved in the solution;
(c) while continuing mixing, adding 300 mg finasteride and 12.5 mg of retinoic acid, until all solids were fully dissolved; and
(d) while continuing mixing, adding 8.6875 mL of room temperature (22° C.-26° C.) water, to complete the hair growth composition.

In such compounding pharmacy compounding of the hair growth composition, minoxidil is added to provide a concentration that is in a range of from 1.2 wt % to 12 wt %, based on total weight of the composition, and acid for pH adjustment, e.g., sulfuric acid, hypochlorous acid (HOCl), citric acid, or other suitable acid, is added only in a quantity required to achieve the specified pH condition of pH 4.5.

While the disclosure has been set forth herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the disclosure as hereinafter claimed is intended to be broadly construed and interpreted, as including all such variations, modifications and alternative embodiments, within its spirit and scope.

What is claimed is:

1. A physician-prescribed and compounding pharmacy-compounded minoxidil-containing hair growth composition, comprising minoxidil, glycerin, polysorbate-80, and propylene glycol in aqueous ethanol solution at pH in a range of from 3.8 to 5.2.

2. The composition of claim 1, which is stable against minoxidil precipitation for at least 6 months at temperature in a range of from 5° C. to 32° C.

3. The composition of claim 1, wherein the physician-prescribed minoxidil concentration is a concentration that is in a range of from 1.2 wt % to 12 wt %, based on total weight of the composition.

4. The composition of claim 1, wherein the physician-prescribed minoxidil concentration is a concentration that is in a range of from 2 wt % to 8 wt %, based on total weight of the composition.

5. The composition of claim 3, wherein the physician-prescribed minoxidil concentration is based on assessment of patient phenotypic and physiological characteristics of a patient, including age, gender, and extent and duration of hair loss.

6. The composition of claim 1, as packaged with topical dosing instructions on or in packaging.

7. The composition of claim 1, further comprising at least one selected from the group consisting of finasteride, dutasteride, retinoic acid, and tretinoin.

8. The composition of claim 1, further comprising at least one of finasteride or dutasteride.

9. The composition of claim 1, further comprising retinoic acid.

10. The composition of claim 1, further comprising tretinoin.

11. The composition of claim 1, wherein pH of the composition is in a range of from 4 to 5.

12. The composition of claim 1, wherein:
glycerin is present at concentration in a range of from 8 wt % to 12 wt %, based on total weight of the composition;
propylene glycol is present at concentration in a range of from 25 wt % to 35 wt %, based on total weight of the composition;
polysorbate-80 is present at concentration in a range of from 6 wt % to 10 wt %, based on total weight of the composition;
ethanol is present at concentration in a range of from 15 wt % to 25 wt %, based on total weight of the composition; and
water is present at concentration in a range of from 22 wt % to 33 wt %, based on total weight of the composition.

13. The composition of claim 1, further comprising acid selected from the group consisting of sulfuric acid, hypochlorous acid, citric acid, and combinations thereof.

14. The composition of claim 13, wherein the acid is present at concentration in a range of from 0.05 wt % to 0.8 wt %.

15. The composition of claim 1, further comprising retinoic acid, tretinoin, and at least one of finasteride and dutasteride.

16. The composition of claim 15, further comprising at least one of sulfuric acid, hypochlorous acid, and citric acid.

17. A method of preparing a hair growth composition, comprising the sequential steps of:
(a) mixing a first amount of water with glycerin, propylene glycol, polysorbate-80, and ethanol, to form a first solution;
(b) heating the first solution under mixing conditions up to a first temperature in a range of from 70° C. to 80° C.;
(c) while maintaining the mixing conditions and heating to maintain the first temperature, adjusting pH of the first solution to pH in a range of from 4 to 5 by addition of acid;
(d) while maintaining the mixing conditions and heating to maintain the first temperature, adding minoxidil to the pH-adjusted first solution until the minoxidil is dissolved, to form a second solution; and
(e) terminating the heating, and while maintaining the mixing conditions, adding a second amount of water at temperature in a range of from 15° C. to 30° C. to the second solution, to yield the composition.

18. The method of claim 17, wherein the acid comprises at least one of sulfuric acid, hypochlorous acid, and citric acid.

19. The method of claim 17, further comprising adding in step (d) at least one selected from the group consisting of finasteride, dutasteride, and tretinoin.

20. The method of claim 17, wherein minoxidil is added to provide a concentration that is in a range of from 1.2 wt % to 12 wt %, based on total weight of the composition.
